(19) United States
(12) Patent Application Publication
(10) Pub. No.: US 2010/0054565 A1
(43) Pub. Date: Mar. 4, 2010

(54) PARALLEL BEAM LOCAL TOMOGRAPHY RECONSTRUCTION METHOD

(75) Inventors: Eric Todd Quinto, Medford, MA (US); Ozan Öktem, Sundbyberg (SE)

Correspondence Address: YOUNG & THOMPSON, 209 Madison Street, Suite 500, Alexandria, VA, 22314, US

(73) Assignee: SIDEC TECHNOLOGIES AB, Kista (SE)

(21) Appl. No.: 12/444,501

(22) PCT Filed: Oct. 8, 2007
(86) PCT No.: PCT/SE07/50719

§ 371 Date: Jul. 8, 2009

Related U.S. Application Data
(60) Provisional application No. 60/849,772, filed on Oct. 6, 2006.

(51) Int. Cl. G06K 9/00; A61B 6/03

(52) U.S. Cl. 382/131; 378/4

(57) ABSTRACT

A method to image objects from local three-dimensional parallel beam tomographic data (line integrals) over lines parallel an arbitrary curve of directions on a sphere. Such data are used in electron microscopy, SPECT (with weighted integrals), and synchrotron tomography. The algorithm is adaptable to a number of data sets including single-axis and double-axis tilt electron tomography and truly three-dimensional curves of directions. The method stably gives pictures of the internal structure of objects and does not add strong singularities or artefacts. It is less influenced by objects outside the region of interest than standard non-local methods. The algorithm is combined with an electron microscope and computer to provide computer readable files showing the pictures of small objects such as molecules.

This invention was made with government support at Tufts University under grants DMS 0200788 and 0456868 and awarded by the United States National Science Foundation. The government has certain rights in the invention.

### TECHNICAL FIELD

The present invention relates to an image analysis and image enhancement method and in particular for use in electron tomography, SPECT and other applications in tomography.

### BACKGROUND OF THE INVENTION

Tomography allows scientists to see the inside structure of objects by probing the object with particles from different directions. Typically, in X-ray transmission tomography photons are used as a probe and the object can be a human being. There are a number of applications where tomographic data are collected over parallel lines in a finite set of directions on a curve, and the lines pass through only a small sub region, the region of interest (ROI), of the object. Such problems are loosely referred to as local tomography problems.

One example is electron microscope tomography (ET) where a transmission electron microscope probes a small sub region of the specimen (i.e. the object) with electrons. Next, the specimen is rotated to other “tilts” or angles and new images are taken of that small sub-region. After some pre-processing, the data can approximately be interpreted as projections of the sub region of the specimen from a finite set of angles. A similar situation appears in synchrotron tomography, where a thin beam of parallel X-rays are used to probe an object. Data are collected and the object is rotated over a range of angles. Another case that involves Single Photon Emission Tomography or SPECT (the particles that probe the object are inside it) [10, 16, 17].

There is no pre-existing algorithm for the specific local problems we consider. Standard reconstruction methods for parallel beam data do not give satisfying results because, among other reasons, they require tomographic data over all lines through the entire object. Therefore, they do not directly apply to the problems we consider, since data in these problems are only over a small subset of lines passing through the ROI. As a result, if one uses the standard algorithms, the data must be extended arbitrarily, and reconstructions (pictures) from the algorithms can blur boundaries and cause distortions. The other type of three-dimensional tomography-cone beam tomography-does not use the same data, so cone beam algorithms do not apply to the problems our algorithm solves.

There are several local tomographic methods, each of which depends on the type of data used. The most well-developed local tomographic methods are for standard CT data for lines in a plane. Lambda CT is the most popular local tomographic method for planar data [3,2,13]. It is the closest planar method to our 3-D method, because it takes a derivative filter of the data. Other methods for planar CT data include pseudo-local tomography [7] (a local version of the standard inversion formula) and wavelet methods [12]. These algorithms are different from our algorithm in two important ways. First, they apply only to data over lines in a plane. Our algorithm applies to a range of data collection schemes. Our data are over lines in a region in space.

Like our algorithm, they are ROI algorithms. However, they assume that data are given over all lines in the ROI. Our algorithm is designed for lines in a limited range of directions (e.g., the curve of directions can be part of a circle, Section 4.3, or an arbitrary arc on the sphere). Kuchment, Lancaster, and Mogeleskaya [8] have adapted Lambda CT to limited angle data sets in the plane. This is closer to our method, but like Lambda CT, it uses only lines in a plane. Also, they do not seem to include the factor μ in their two-dimensional method. This factor significantly enhances objects in the reconstruction since it adds density information (see [2] for a justification for two-dimensional Lambda CT). Louis and Maaβ patented a local tomography method for three-dimensional cone-beam tomography. Their method is different from ours in two ways. First, their method is for a different data set-cone beam data-that is three dimensional X-ray CT data from lines that pass through a curve. Our data set is completely different since it includes only lines parallel a cone rather than lines through a curve. They use a derivative filter in the detector plane, but their derivative filter is different from ours (it takes derivatives (the Laplacian) in two directions). Katsevich [5], Anastasio, et al. [1], and Ye et al. [14], have developed refinements of this algorithm that uses a derivative more like ours (in one direction). Their methods are closer to ours than the Louis and Maaβ method. However, the geometry of their data collection scheme—cone beam geometry—is different from ours; Therefore, all of these methods do not apply to our problems.

Several authors have developed methods for complete slant hole SPECT data. Such data are parallel beam tomographic data when the curve of directions, C, is a latitude circle on the sphere. Their methods all require complete data (data over all lines parallel the curve), and our method requires only local data (data only over lines in a region of interest). Some time ago, Orlov [15] developed an inversion method for the parallel beam transform (without a weight) and for this latitude circle C if all parallel beam data are used, but it does not work with the region of interest data as in our case. For slant hole SPECT data, Clackdoyle [16] and Kunyansky [17] have recently developed inversion methods that require all slant hole data and an exponentially weighted parallel beam transform. None of these methods apply to our problem because they require complete data, not just data through a region of interest.

### SUMMARY OF THE INVENTION

It is an object of the present invention to remedy at least some of the problems with the existing technologies and provide an algorithm and method for increasing the resolution and effective information in resulting images taken in tomography measurements using parallel beam data with angles on a curve.

This invention provides a new method to image objects from local three-dimensional parallel beam tomographic data (line integrals) over an arbitrary curve of directions. Such data are used in electron microscopy, synchrotron tomography, and SPECT. The algorithm is adaptable to a number of data sets including single-axis and double-axis tilt electron tomography and truly three-dimensional curves of directions. The method stably gives pictures of the internal structure of objects and does not add singularities or artefacts, as other methods can. It is less influenced by objects outside the region of interest than standard nonlocal methods. The algorithm is combined with a data collector (e.g., an electron microscope) and computer to provide computer readable files showing the pictures of small objects such as molecules.

The present invention is realized in a number of aspects in which a method to reconstruct the function representing an unknown entity from local three-dimensional parallel beam tomographic data, i.e. data can (after possible pre processing) be interpreted as samples of the parallel beam transform (or its weighted version) restricted to lines parallel a curve C of directions and passing through a pre-specified region of interest, comprising the steps of:
derivative filtering in the detector plane;
smoothing in the detector plane in the perpendicular direction thereby averaging the data in each detector plane using data from neighbouring detector planes; and
applying the corresponding back projection operator, e.g. using equations (8) or (8b) depending on whether data can be reinterpreted as samples of the parallel beam transform or its weighted version, and then applying post-processing.

The method may further comprise the steps of:
obtaining data (e.g., from an electron microscope from several tilts);
transferring the data to a computer;
optionally pre processing the data to transform it into tomographic projection data;
running a computer program implementing the method as discussed above;
producing in the computer a computer readable file stored on a storage media of the reconstruction of the object.

The method may be arranged to receive three-dimensional parallel beam tomographic data with directions on a curve: examples of modalities that yields such data are data from electron microscopy, synchrotron data, x-ray data (e.g. mammography data), slant-hole SPECT tomography.

The method may be applied locally, i.e. it uses only lines near a point to reconstruct at the point:
it needs only data in a region of interest to reconstruct that region of interest.
a dense object outside the region can have less influence on the reconstruction than it would on standard reconstruction methods that use all data through the entire object, including the dense object.

The method may be arranged to efficiently handle limited data sets, i.e. some parts of the objects are not reliably visible from the data or some features of the objects can be difficult to image from the data.

The data sets the method uses may be in a limited range of directions, i.e. the curve C may be an arbitrary continuous curve on the sphere.

The method may emphasize boundaries of objects in the reconstruction so that the boundaries are easier to see because of the derivative filter; it de-emphasizes added artefacts and it does not distort object boundaries that are not stably visible from the data.

The method may be arranged to apply to a large range of parallel beam data acquisition geometries by altering the curve and choosing the ROI (region of interest).

A second aspect of the present invention, a data ensemble is provided, comprising image information wherein the image information has been derived using the method as claimed in the patent claims.

Yet another aspect of the present invention, a computer readable storage media containing the data ensemble, may be provided.

Still another aspect of the present invention, a signal relating to the data ensemble for transportation in a communication network is provided,

This invention was made with government support at Tufts University under grants DMS 0200788 and 0456868 and awarded by the US National Science Foundation. The government has certain rights in the invention.

### BRIEF DESCRIPTION OF THE DRAWINGS

In the following the invention will be described in a non-limiting way and in more detail with reference to exemplary embodiments illustrated in the enclosed drawings, in which:

FIG. 1 illustrates schematically a general method according to the present invention;

FIG. 2 illustrates schematically a system for implementing the present invention;

FIG. 3 illustrates schematically a device for using the present invention;

FIG. 4 illustrates image result from the method illustrated in FIG. 1 applied to electron tomography compared to known technology; and

FIG. 5 illustrates image result from the method illustrated in FIG. 1 applied to electron tomography compared to known technology;

### DETAILED DESCRIPTION OF PREFERRED EMBODIMENTS

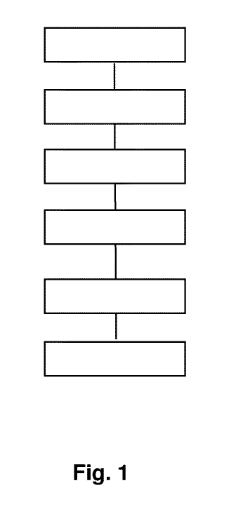

FIG. 1 illustrates the method where the reference numerals indicated is the same as below step numbers:
1. Prepare object to be imaged: The preparation depends on the type of object and imaging device that is to be used (i.e. the imaging modality) and should be understood by the persons skilled in the art. In the case of electron tomography, the object is an in-vitro or in-situ biological sample and the imaging device is a transmission electron microscope with a specimen holder that can rotate.
2. Mount the object into the imaging device: The mounting procedure may vary depending on the imaging device that is to be used and also this should be understood by a person skilled in the art.
3. Using the imaging device, collect tomographic parallel beam data of the object. A detector included in the imaging device converts each image to a computer file which is transferred to the computer. In the case of electron tomography, the electron microscope takes about 60-120 images, rotating the specimen in a range of roughly ±60° about a fixed axis. In the case of slant-hole SPECT, the SPECT scanner can take data over lines with directions on a latitude circle (often at roughly 45°).
4. Pre-process the tomographic data as to represent line integrals of the function representing the structure of the object to be reconstructed. Moreover, the data in the computer file is altered to become data “in the same coordinate system” so it can be reconstructed. In the case of electron tomography, data is rescaled and aligned. The latter is the term used for the procedure to more precisely determine the actual data collection geometry. For electron microscopy, as the specimen is rotated in the microscope, due to the small measurement scale there are unintentional movements that must be corrected. Common alignment procedures use gold markers in the specimen to determine the actual measurement geometry.
5. A computer reads in the pre-processed computer data file and processes it. There are two versions of the algorithm itself, as described in Section 4.1, Version 4.1.A with less post-processing and Version 4.1.B with more. Both take the data and average it and then sum up the averaged data to get the reconstruction. Note that versions of the algorithm are described for both parallel beam and attenuated (SPECT) data.
6. In each version, the algorithm creates a computer file representing a three-dimensional picture of the reconstruction of the object (voxel elements are values of the reconstruction of the object in each voxel). After thresholding, the picture shows the shapes of the structure in the object imaged by the imaging device. This file can be used for further interpretation by a person skilled in the art.

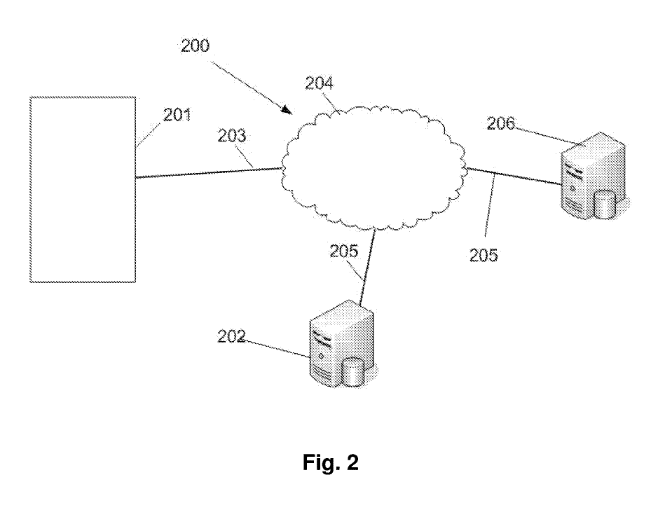

In FIG. 2 reference numeral 200 generally denotes an imaging device (e.g. an electron microscope) with an image acquisition device 201 with a detecting device (not shown) connected 203, 205 to a processing device 202 directly or indirectly through a network 204. Data from the image acquisition device is transmitted to the processing device 202 that also may be arranged to control the configuration and operation of the image acquisition device 201. The image acquisition device 201 are known to the person skilled in the art and therefore not described further in this document. Data obtained in the processing device may be transmitted to other devices such as an external PC 206 connected 205 to the same network 204. In the setup for electron tomography, the image acquisition device 201 is a transmission electron microscope.

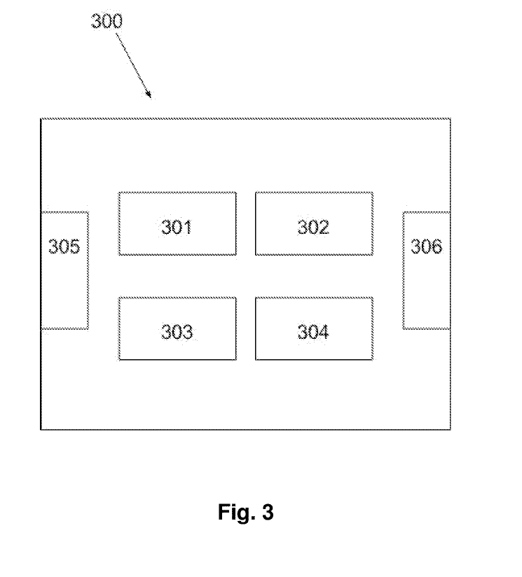

The processing device 202, 300 is shown in detail in FIG. 3, wherein a processing unit 301 handles image analysis and interaction with the imaging device and user. The processing device 300 further comprises a volatile (e.g. RAM) 302 and/or non volatile memory (e.g. a hard disk or flash disk) 303, an interface unit 304. The processing device 300 may further comprise a data acquisition unit 305 and communication unit 306, each with a respective connecting interface. All units in the processing device can communicate with each other directly or indirectly through the processing unit 301. The processing unit 301 processes data, controls data acquisition, and handles interface commands using appropriate software, data and analysis results may be stored in the memory unit(s) 302, 303. The interface unit 304 interacts with interface equipment (not shown), such as input devices (e.g. keyboard and mouse) and display devices. The data acquisition unit 305 interacts with the image acquisition device 201 and receives data from the image acquisition device. The communication unit 306 communicates with other devices via for instance a network (e.g. Ethernet). Image data can also be stored and analyzed later in the processing device 300 or in any other suitable processing device, e.g. a server, personal computer or workstation. The analysis method according to the present invention is usually realized as computer software stored in the memory 302, 303 and run in the processing unit 301. The analysis software can be implemented as a computer program product and distributed on a removable computer readable media, e.g. diskette, CD-ROM (Compact Disk-Read Only Memory), DVD (Digital Video Disk), flash or similar removable memory media (e.g. compactflash, SD secure digital, memorystick, miniSD, MMC multimediacard, smartmedia, transflash, XD), HD-DVD (High Definition DVD), or Bluray DVD, USB (Universal Serial Bus) based removable memory media, magnetic tape media, optical storage media, magneto-optical media, bubble memory, or distributed as a propagated signal via a computer network (e.g. Internet, a Local Area Network (LAN), or similar networks). The same type of media may be used for distributing results from the measurements of the electron microscope for post analysis at some other computational/processing device. It should be understood that the processing device may be a stand alone device or a device built into the imaging device. It may alternatively be a device without possibilities to control the image acquisition device and used for running image analysis, e.g. the external PC 206.

Tomographic data are pre-processed as to represent line integrals of the function representing the structure of the object, which in the case of electron tomography is the electrostatic potential of molecules in the sample. The type of pre-processing and geometric distribution of the lines is determined by the experimental setup, the sample, and the detector that is used. In a formal sense, the data are assumed to be line integrals of some property of the object to be imaged. Other types of pre processing may also be done on the experimental data.

The present invention includes a new method to process three-dimensional parallel beam tomographic data to get better images of small objects when only a sub region of the object is probed. This algorithm is useful for many problems, including electron microscopy, synchrotron tomography, and SPECT. The algorithm is local in that it needs only data through a region of interest (ROI) to image that region. Furthermore, the algorithm does not need to use all lines through the region but only those in a limited range of angles in space, and therefore the algorithm solves several limited data problems. As a result, objects (such as gold markers) outside the region of interest have less effect on the reconstruction than with the standard methods which are not local.

The present invention is an algorithm and virtual machine to provide high-quality reconstructions (pictures) from parallel beam tomographic data over lines that go through a small region of interest in an object in a limited range of directions in space. In our data collection scheme, the directions are on a given curve. Such data are acquired from electron microscope with tilts (electron tomography (ET)), synchrotron tomography, and slant hole SPECT [10] (with weighted line integrals). In each case, data can either directly be interpreted or after preprocessing, be interpreted as (perhaps weighted) line integrals of an unknown function representing a property to be reconstructed.

The present invention processes the data in each detector plane (for each image for each fixed direction) in a novel way, taking a derivative filter in one direction and smoothing in the other ((7) or (7b)). Then, the invention back projects or averages the data. For each point in the region of interest, the back projection step averages data over lines in the data set near that point to get the final picture (see (9) and (9b)). Thus, the method is designed for region of interest tomography, where only a small region in the object is imaged and only data going through that object are used. Because the problems we consider are limited data, some details (e.g., boundaries) of the objects are not reliably visible from the data [9]. The processing on the detector plane (the smoothing step) ensures that our method does not add singularities as other methods can [11] ([5,6,4] show similar added singularities in cone beam CT). Because of the derivative filter, the algorithm emphasizes boundaries of objects so that the boundaries are easier to see.

The method applies to a large range of data acquisition geometries not covered by the existing algorithms. It is easy to adapt to each of these situations by altering the curve and choosing the ROI.

The algorithm substantially includes three steps:
1. a derivative along the lines (plus an additive factor of μ).
2. smoothing in the detector plane the perpendicular direction to the derivative,
3. using a back projection operator.

The details will now be given. Let S² be the unit sphere in space, R³ is the three-dimensional Euclidian space. For ω ∈ S², let

ω^{⊥}:={x ∈ R³|x·ω=0} (1)

be the plane through the origin perpendicular to ω. This will be called the detector plane even though the plane of detectors is typically only parallel this plane.

The reconstruction problem can be recast as a reconstruction from parallel beam tomographic data, for example after the original data has been preprocessed. The result can be assumed to be the parallel beam X-ray transform:

P(f)(y,ω):=∫_{-∞}^{∞}f(y+tω)dt for ω ∈ S² and y ∈ ω^{⊥}. (2)

This is simply the integral of f over the line

l(y,ω):={y+tω|t ∈ R}. (3)

The transform in slant-hole SPECT is the attenuated X-ray transform, a weighted parallel beam transform [10]:

Eᵥ(f)(y,ω):=∫_{-∞}^{∞}f(y+tω))e^{vt}dt for ω ∈ S² and y ∈ ω^{⊥}. (2b)

Let C be the curve of directions on S² used for the given data set. We let

Y:={(y,ω)|ω ∈ C, y ∈ ω^{⊥}}. (4)

Then Y represents the set of all lines with direction parallel to C or equivalently, the set of lines parallel the cone generated by C. The data set is a subset of Y.

The derivative filter we use is defined as follows. Assume the curve C is parameterized by the differentiable function ω(θ) with ω′(θ)≠0 and let

$$σ ( θ ) := \frac{{ω}^{′} ( θ )}{\vert {ω}^{′} ( θ ) \vert} \qquad ( 5 )$$

be a unit tangent to the curve C at ω(θ). Then, we denote the second derivative in direction σ by

$${{D}_{σ}^{2} g ( y , ω ( θ ) ) := \frac{{d}^{2}}{d {s}^{2}} g ( y + s σ ( θ ) , ω ( θ ) ) \vert}_{s - 0} . \qquad ( 6 )$$

Note that there is a negative sign in front of the derivative in (6). The negative sign makes this operator consistent with [2,3] and, when combining with μ in (9) or (9b) below shows regions better (see [2,3] for a justification for planar Lambda CT).

For very noisy data, such as electron microscopy data, we smooth in two ways. First, we take the derivative D_{σ}² using a kernel that is a smoothed version of the second derivative. Second, we smooth by averaging nearby slices, that is, we also convolve in the plane ω^{⊥} in the direction perpendicular to σ. Let K be a one-dimensional approximate identity. For us, this means that K is even, ∫K(x)dx=1 and K ∈ S(R). We let [symbol] denote the one dimensional convolution in the plane ω^{⊥} in direction perpendicular to σ. After this processing, for each ω ∈ C, the filtered data on the detector plane ω^{⊥} is

$$( K {*}_{x} ( {D}_{σ}^{2} + μ ) P ) ( f ) ( y , ω ) \qquad ( 7 )$$

where y is in the detector plane ω^{⊥} and μ is a constant. We include the factor μ, as is done for standard Lambda tomography, to provide contour to the reconstruction.

The filtering in the detector plane for SPECT is

$$( K {*}_{x} ( {D}_{σ}^{2} + μ ) {E}_{v} ) ( f ) ( y , ω ) \qquad ( 7 b )$$

The back projection operator for P is the dual parallel beam transform for angles on the curve C,

$${P}_{C}^{*} g ( x ) := {\int }_{ω εC} g ( x - ( x \cdot ω ) ω , ω ) d ω \qquad ( 8 )$$

where the measure dω is the arc length measure on the curve C and the point x−(x·ω)ω is the projection of x onto the plane ω^{⊥}. P_{C}*g is the average of g over all lines through x and parallel to C. If the curve C is not closed, as in single-axis tilt ET, we taper the weight of integration at the ends of the curve (see (15)).

The back projection operator for SPECT is

$${E}_{- v}^{*} g ( x ) := {\int }_{ω εC} g ( x - ( x \cdot ω ) ω , ω ) {e}^{- vx \cdot ω} d ω \qquad ( 8 b )$$

Our reconstruction operator for P, the parallel beam transform, is

$$L ( f ) ( x ) := {P}_{C}^{*} ( K {*}_{x} ( {D}_{σ}^{2} + μ ) P ) ( f ) ( x ) \qquad ( 9 )$$

where μ is a constant. Professor Quinto has proven that L is a pseudo-differential operator with a mildly singular symbol. L adds weaker singularities than P_{C}* Δ P and the added singularities are intrinsic to the problem. They are in a particular direction (or set of directions) that is different from the singularities that are visible in the data. The point of using L rather than P_{C}* Δ P is to de-emphasize these added singularities (see [10,11] for a complete discussion). In basic terms, this says that L will not add strong singularities to the reconstruction and these singularities are in a special direction.

The reconstruction operator for SPECT becomes

$${L}_{v} ( f ) ( x ) := {E}_{- v}^{*} ( K {*}_{x} ( {D}_{σ}^{2} + μ ) {E}_{v} ) ( f ) ( x ) \qquad ( 9 b )$$

The negative sign in E₋ᵥ* is included so that the operator Lᵥ is simpler [10].

Now, we can see clearly why the algorithm is a ROI algorithm. To calculate the result at x one needs only to know the value of P f (or Eᵥ(f)) over lines near x because, to calculate the filtered data on the detector plane (7) one needs only data P f over lines near x, and the back projection needs only these lines to calculate the final result, L(f)(x) (and similarly for Lᵥ)

4.1. The Algorithm

There are two versions of the algorithm, depending on post-processing. They are described for the parallel beam transform and the application to SPECT is the same except one replaces P by Eᵥ and P* by E₋ᵥ*.

Version A: Less Post-Processing.
1. Take an averaging filter of the data in the perpendicular direction to σ(θ) in ω(θ)^{⊥}, getting K [symbol] P f.
2. Take the filtered data from step 1 and take back projection. This gives f_{b}(x):=P_{C}*(K [symbol] P f).
3. Take the filtered data from step 1 and filter again using an averaged derivative in the σ(θ) direction (getting K [symbol] D_{σ}² P f). Then, take back projection. This gives f_{d}(x):=P_{C}*(K [symbol] D_{σ}² P f).
4. Let μ≧0. The reconstruction is f_{d}+μf_{b}. This is exactly L(f).
5. Do post-processing and scaling and display the results. One type of post processing truncates the negative values of the reconstruction.

Version B: More Post Processing.
1. Do steps 1, 2, and 3 from Version A. This gives the functions f_{b} and f_{d}.
2. Linearly alter the range of f_{b} and f_{d} so that their minima are both equal to zero and their maxima are equal to one. Then, take a convex combination of f_{b} and f_{d}, the reconstruction is cf_{b}+(1−c)f_{b}.
3. Display the results.

This second version is useful when there is not much contrast in f_{b}, which can happen in ET.

4.2. Flowchart of Invention

The system includes an imaging device with a digital detector connected to a computer. The computer is programmed with the algorithm outlined in Section 4.1 and it is connected to a display. The result of the invention is a computer file that shows the inner structure of the object being scanned by the imaging device. The method may be illustrated as shown in FIG. 1, where the reference numerals indicated is the same as below step numbers:
1. Prepare object to be imaged: The preparation depends on the type of object and imaging device that is to be used (i.e. the imaging modality) and should be understood by the persons skilled in the art. In the case of electron tomography, the object is an in-vitro or in-situ biological sample and the imaging device is a transmission electron microscope with a specimen holder that can rotate.
2. Mount the object into the imaging device: The mounting procedure may vary depending on the imaging device that is to be used and also this should be understood by a person skilled in the art.
3. Using the imaging device, collect tomographic parallel beam data of the object. A detector included in the imaging device converts each image to a computer file which is transferred to the computer. In the case of electron tomography, the electron microscope takes about 60-120 images, rotating the specimen in a range of roughly ±60° about a fixed axis. In the case of slant-hole SPECT, the SPECT scanner can take data over lines with directions on a latitude circle at roughly 45°.
4. Pre-process the tomographic data as to represent line integrals of the function representing the structure of the object to be reconstructed. Moreover, the data in the computer file is altered to become data “in the same coordinate system” so it can be reconstructed. In the case of electron tomography, data is rescaled and aligned. The latter is the term used for the procedure to more precisely determine the actual data collection geometry. In the case of electron microscopy, as the specimen is rotated in the microscope, due to the small measurement scale unintentional movements occur that must be corrected. Common alignment procedures use gold markers in the specimen to determine the actual measurement geometry.
5. In the case of electron microscopy, a computer reads in the aligned computer data file and processes it. There are two versions of the algorithm itself (both for the parallel beam transform and for SPECT), as described in Section 4. 1, Version 4.1.A with less post-processing and Version 4.1.B with more. Both take the data and average it and then sum up the averaged data to get the reconstruction.
6. In each version, the algorithm creates a computer file representing a three-dimensional picture of the reconstruction of the object (voxel elements are values of the reconstruction of the object in each voxel). After thresholding, the picture shows the shapes of the structure in the object imaged by the imaging device. This file can be used for further interpretation by a person skilled in the art.

4.3. Example of the Invention Applied to Single- or Dual-Axis Tilt ET

In electron microscope tomography (ET), the imaging device is a transmission electron microscope and the object is a thin (approx. 100 nm thick) biological specimen. The specimen is placed in the microscope and a thin electron beam penetrates a small region in the object. Under appropriate assumptions and pre-processing, the data collected represent line integrals over the lines the electrons traverse of some property of the object that indicates its shape. Data are taken as the object is rotated through different angles. The goal in ET is to reconstruct the shape of individual molecules, each of which can be fairly arbitrary, in-situ or in-vitro. Because the object is much longer than the electron beam is wide, the beam covers only a small region of interest (ROI) in the object. Because the object is long, one can rotate the beam in only a limited range of angles, the problem is a limited angle problem. These imply that one cannot exactly reconstruct the shape of the object. Furthermore, the data are very noisy, in particular because of the dose problem-if the dose is too high, the object is destroyed.

In single-axis tilt electron tomography, one restricts the angles to a single tilt axis, and the curve C becomes an arc of a great circle on S². We use a coordinate system where the electrons come in along the z-axis when ω=(0,0,1) and we assume the tilt axis is the x-axis. Because the object cannot be rotated fully, this means that the curve of directions, C, is an arc of a circle in the (y,z)-plane and there is a limited angular range of ±θₘₐₓ where θₘₐₓ≈2π/3 radians. So, the appropriate parameterization for the curve C in this setting is

ω(θ):=(0, sin θ, cos θ), θ ∈ [−θₘₐₓ,θₘₐₓ] (10)

and

σ(θ):=(0, cos θ,−sin θ), (11)

Now, e₁:=(1,0,0) and σ(θ) form an orthonormal basis of the plane ω(θ)^{⊥}, and they provide orthonormal coordinates on ω(θ)^{⊥}:

y=(y₁,y_{σ}) [symbol] y₁e₁+y_{σ}σ(θ)∈ ω(θ)^{⊥}. (12)

In these coordinates, functions on lines will be written g(y,θ)=g((y₁,y_{σ}),θ) so P has parameterization

P(f)(y,θ):=P(f)(y₁e₁+y_{σ}σ(θ),ω(θ)) (13)

and the set of lines is parameterized by

Y={(y,θ)|y=(y₁,y_{σ})∈ R²,θ ∈]−θₘₐₓ,θₘₐₓ[}(y,θ) [symbol] l(y,θ):=l(y₁e₁+y_{σ}σ(θ),ω(θ)) (14)

We smooth the dual operator P_{θ}_{ₘₐₓ}* slightly so that the numerical integration is more accurate. We choose a non-negative, smooth, function φ:[−π/2,π/2]→[0,1] that is supported in [−θₘₐₓ,θₘₐₓ] and equal to one on most of this interval and extend it to R making it π-periodic. The smoothed limited angle dual parallel beam transform is defined by

$${P}_{{θ}_{max}}^{*} ( g ) ( x ) := {\int }_{- {θ}_{max}}^{{θ}_{max}} g ( x - ( x \cdot ω ( θ ) ) ω ( θ ) , ω ( θ ) ) ϕ ( θ ) d θ . \qquad ( 15 )$$

The expression x−(x·ω(θ))ω(θ) in (15) is the projection of x onto the detector plane ω^{⊥}. We found that a simple trapezoidal rule integration works best in (15); this corresponds to a specific choice of φ.

In coordinates, D_{σ}² becomes

$${{D}_{σ}^{2} g ( y , θ ) := \frac{{d}^{2}}{d {s}^{2}} g ( ( {y}_{1} , s ) θ ) \vert}_{s = 0} . \qquad ( 16 )$$

As in general, we smooth by averaging nearby slices. For single-axis tilt ET, this means that we convolve in the x-variable. Let K(x) be a one-dimensional smooth approximate identity. For single-axis tilt, [symbol] denotes the one-dimensional convolution in the x-variable.

The resulting operator is

$$L ( f ) ( x ) := {P}_{{θ}_{max}}^{*} ( K {*}_{x} ( {D}_{σ}^{2} + μ ) ) P f ( x ) . \qquad ( 17 )$$

Professor Quinto has proven that L is a mildly singular pseudo-differential operator if K is chosen properly [11] and [10] in general.

For dual-axis tilt ET, one just averages the resulting operators for each tilt axis. This is the same method as described for the general case in section 4 where the curve C is now the union of the two circular arcs defining the tilts in each direction.

If the electron microscope scans by holding the object at a specific angle from the z-axis and rotating around the z-axis, then the curve C would be a latitude circle on the sphere, and the general algorithm would apply to this type of ET, too, just using this curve C. This is the data in slant hole SPECT [16,17].

Examples of use of the present invention in electron microscopy will now be discussed.

In-vitro Sample

Study Objective

This set of electron tomography (ET) data was collected within an academic collaboration between the Karolinska Institute, Karolinska Hospital and Huddinge University Hospital in Sweden. The objective was to study the conformation and flexibility of monoclonal IgG molecules with a molecular weight of 150 kDa.

Specification of the ET Data Collection

The specimen is in-vitro monoclonal IgG2a at 0.5 mg/ml with 10 nm gold coated with BSA (washed to remove unbound BSA). ET data collection was done in a 200 kV transmission electron microscope (TEM) at 1 μm under focus with a dose of 1820 e⁻/nm². The tilt series was collected from single axis tilting with a uniform sampling of the tilt angle in [−60°,60°] with 1° step. The pixel size is 0.5241 nm.

Reconstructions

The reconstruction region, which is the region used to collect a tilt-series from, is 256×256×256 pixels in size. Our local region of interest is centered in the mid point of the reconstruction region and is of 128×128×128 pixels size.

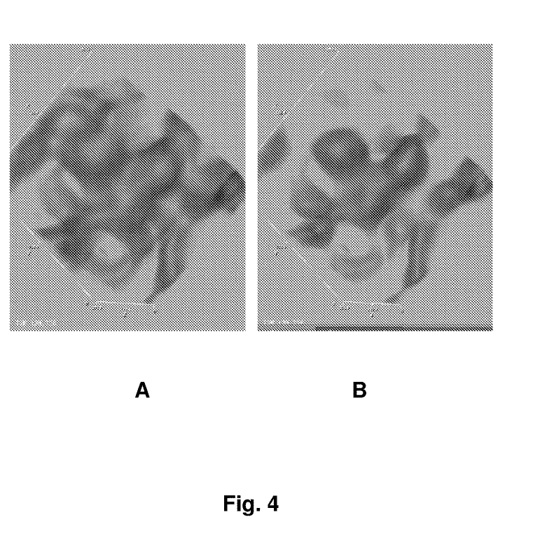

FIG. 4 shows both the low-pass filtered (10 nm) back projection (low-pass FBP) reconstruction and the limited angle Lambda reconstruction. The low-pass FBP is applied to the entire reconstruction region and the region of interest is then been extracted. The limited angle Lambda reconstruction is applied directly on the region of interest. It is clear that the background noise is suppressed in the latter and the IgG molecule (which is in the center) is clearly visible. FIG. 4A shows the low-pass FBP reconstruction and FIG. 4B shows the limited angle Lambda reconstruction.

In-situ Tissue Sample

Study Objective

This set of electron tomography (ET) data was collected within an academic collaboration between the University of Helsinki, Karolinska Institute, University of Oulo and University of California Davis. The objective was to study the role of the slit diaphragm in protein filtration in order to obtain valuable insights into the disease mechanisms of proteinurea.

Specification of the ET Data Collection

In situ tissue sample (could be human, rat or mice kidney) that is in epoxy resin using standard fixation, embedding, and preparation methods and cryosectioned after immunostaining. ET data collection was done in a 200 kV transmission electron microscope (TEM) at 1 μm under focus with a dose of 1500-2000 e⁻/nm². The tilt series was collected from single axis tilting with a uniform sampling of the tilt angle in [−60°,60°] with 2° step. The pixel size is 0.5241 nm.

Reconstructions

The reconstruction region, which is the region used to collect a tilt series from, is 300×300×150 pixels in size. Our local region of interest is a box inside the reconstruction region with a size of 250×200×140 pixels.

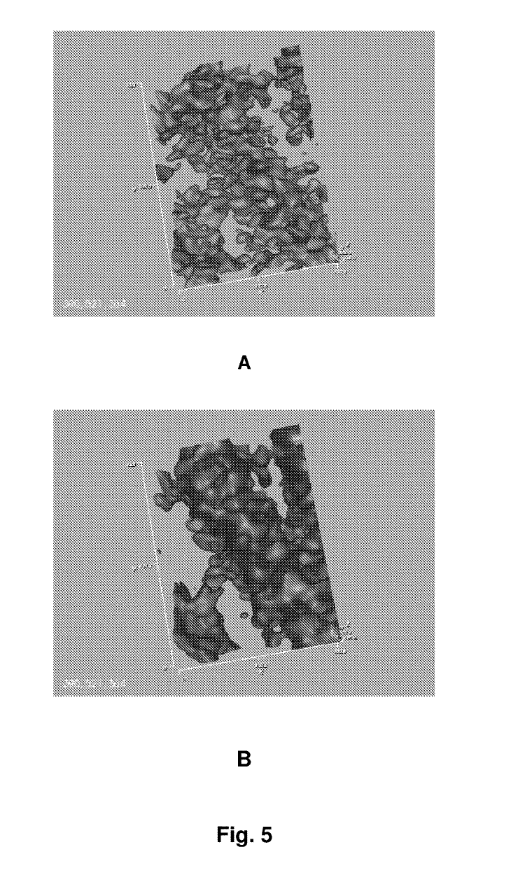

FIG. 5 shows both the low-pass filtered (10 nm) back projection (low-pass FBP) reconstruction and the limited angle Lambda reconstruction. The low-pass FBP is applied to the entire reconstruction region and the region of interest is then been extracted. The limited angle Lambda reconstruction is applied directly on the region of interest. It is clear that the background noise is suppressed in the latter and the “V” shaped region containing the slit diaphragm is more clearly defined. FIG. 5: FIG. 5A shows the low-pass FBP reconstruction and FIG. 5B shows the limited angle Lambda reconstruction.

Even though the present invention has been described in relation to tomographic data it may be applied to other types of image data, such as but not limited to, x-ray data (e.g. mammography images), synchrotron data, and slant-hole SPECT tomography.

The method according to the present invention may be applied locally, i.e. it uses only lines near a point to reconstruct at the point:

it needs only data in a region of interest to reconstruct that region of interest.

a dense object outside the region can have less influence on the reconstruction than it would on standard reconstruction methods that use all data through the entire object, including the dense object.

The method may be arranged to efficiently handle limited data sets, i.e. some parts of the objects are not reliably visible from the data or some features of the objects can be difficult to image from the data.

The data sets the method may use are in a limited range of directions, i.e. the curve C may be arbitrary.

The method may emphasize boundaries of objects so that the boundaries are easier to see because of the derivative filter; it de-emphasizes added artefacts and it does not distort object boundaries that are not stably visible from the data.

The method may be arranged to apply to a large range of data acquisition geometries by altering the curve and choosing the ROI (region of interest) for each geometry.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, that at least parts of the invention may be implemented by means of both hardware and software, and that several “means”, “units” and “devices” may be represented by the same item of hardware.

The above mentioned and described embodiments are only given as examples and should not be seen to be limiting to the present invention. Other solutions, uses, objectives, and functions within the scope of the invention as claimed in the below described patent claims should be apparent for the person skilled in the art.

### REFERENCES

[1] M. A. Anastasio, Y. Zou, E. Y. Sudley, and X. Pan. Local cone-beam tomography image reconstruction on chords. Technical report, Illinois Institute of Technology, 2006.

[2] A. Faridani, D. Finch, E. L. Ritman, and K. T. Smith. Local tomography, II. SIAM Journal on Applied Mathematics, 57:1095-1127, 1997.

[3] A. Faridani, E. L. Ritman, and K. T. Smith. Local tomography. SIAM Journal on Applied Mathematics, 52:459-484, 1992.

[4] David Victor Finch, Ih-Ren Lan, and Gunther Uhlmann. Microlocal Analysis of the Restricted X-ray Transform with Sources on a Curve. In Gunther Uhlmann, editor, Inside Out, Inverse Problems and Applications, volume 47 of MSRI Publications, pages 193-218. Cambridge University Press, 2003.

[5] A. I. Katsevich. Improved cone beam local tomography. Inverse Problems, 22:627-643, 2006.

[6] Alexander I. Katsevich. Cone beam local tomography. SIAM J. Appl. Math., 59:2224-2246, 1999.

[7] Alexander I. Katsevich and Alexander G. Ramm. Pseudolocal tomography. SIAM J. Appl. Math., 56:167-191, 1996.

[8] P. Kuchment, K. Lancaster, and L. Mogilevskaya. On local tomography. Inverse Problems, 11:571-589, 1995.

[9] E. T. Quinto. Singularities of the X-ray transform and limited data tomography in R² and R³. SIAM Journal on Mathematical Analysis, 24:1215-1225, 1993.

[10] E. T. Quinto, T. Bakhos, and S. Chung. A local algorithm for Slant Hole SPECT. Technical report, Tufts University, 2007. in preparation.

[11] E. T. Quinto and O. Öktem. Local tomography in electron microscopy. Technical report, Tufts University and Sidec Technologies, 2007.

[12] F. Rashid-Farrokhi, K. J. R. Liu, Carlos A. Berenstein, and David Walnut. Wavelet-based multiresolution local tomography. IEEE Trans. Image Processing, 6:1412-1430, 1997.

[13] E. I. Vainberg, I. A. Kazak, and V. P. Kurozaev. Reconstruction of the internal three-dimensional structure of objects based on real-time integral projections. Soviet Journal of Nondestructive Testing, 17:415-423, 1981.

[14] Y. Ye, H. Yu, and G. Wang, Cone beam pseudo-lambda tomography, Inverse Problems, 23(2007), 203-215.

[15] S. S. Orlov, Theory of three dimensional reconstruction. 1. Conditions of a complete set of projections, Sov. Phys.-Crystallogr. 20(1975), 312-314.

[16] J-M Wagner and F. Noo and R. Clackdoyle, Exact Inversion of the exponential X-ray transform for rotating slant-hole (RSH) SPECT, Phys. Med. Biol., 47(2002), 2713-2726.

[17] L. Kunyansky, Inversion of the 3-D exponential parallel-beam transform and Radon transform with angle-dependent attenuation, Inverse Problems, 20(2004), 1455-1478.

1. A method to reconstruct a function representing an unknown entity from local three-dimensional parallel beam tomographic data, i.e. data can (after possible pre-processing) be interpreted as samples of the parallel beam transform (or its weighted version) restricted to lines parallel a curve, C, of directions and passing through a pre-specified region of interest, comprising the steps of:
a) obtaining data from a detector;
b) derivative filtering in the detector plane;
c) smoothing in the detector plane in the perpendicular direction thereby averaging the data in each detector plane using data from neighbouring detector planes; and
d) applying a corresponding back projection operator depending on whether data can be reinterpreted as samples of the parallel beam transform or its weighted version, and then applying post-processing.

2. The method according to claim 1, further comprising the step of:
obtaining data from the detector in an imaging device from several directions along a curve.
optionally pre processing the data to transform it into samples of parallel beam tomographic projection data, wherein the samples of projection data is optionally weighted;
producing a computer readable file stored on a storage media of the reconstruction of the entity.

3. The method according to claim 1, wherein the method is arranged to receive three-dimensional parallel beam tomographic data with directions on a curve: the data can be provided from imaging devices such as data from electron microscopy, synchrotron data, x-ray data (e.g. mammography data), slant-hole SPECT tomography (with weighted line integrals), and other tomographic modalities.

4. The method according to claim 1, wherein the method is applied locally, i.e. it uses only lines near a point to reconstruct at the point.

5. The method according to claim 1, wherein the method is arranged to efficiently handle limited data sets.

6. The method according to claim 1, wherein the data sets the method uses are in a limited range of directions, i.e. the curve C may be arbitrary.

7. The method according to claim 1, wherein the method emphasizes boundaries of objects so that the boundaries are easier to see because of the derivative filter (16); it de-emphasizes added artefacts and it does not distort object boundaries that are not stably visible from the data.

8. The method according to claim 1, wherein the method is arranged to apply to a large range of data acquisition geometries by altering the curve and choosing the ROI (region of interest) for each geometry.

9. A data ensemble comprising image information, wherein the image information has been derived using the method according to claim 1.

10. A computer readable storage media containing a data ensemble according to claim 9.

11. A signal transported in a communication network, relating to a data ensemble according to claim 9.
